(19) United States
(12) Patent Application Publication
(10) Pub. No.: US 2018/0325106 A1
(43) Pub. Date: Nov. 15, 2018

(54) METHOD OF RETARDING AN ETHYLENE RESPONSE

(71) Applicant: CURTIN UNIVERSITY OF TECHNOLOGY, Bentley (AU)

(72) Inventors: Zora SINGH, Bentley (AU); Alan David PAYNE, Bentley (AU); Shamim Ahmed Kamal Uddin KHAN, Bentley (AU); Muftah Miload MUSA, Bentley (AU)

(73) Assignee: CURTIN UNIVERSITY OF TECHNOLOGY, Bentley (AU)

(21) Appl. No.: 15/772,324

(22) PCT Filed: Nov. 3, 2016
(86) PCT No.: PCT/AU2016/051048

§ 371 Date: Apr. 30, 2018

(30) Foreign Application Priority Data
Nov. 3, 2015 (AU) 2015904507

(51) Int. Cl. A01N 27/00; A01N 3/02

(52) U.S. Cl. CPC A01N 27/00; C07C 13/547; A01N 3/02

(57) ABSTRACT

A method for retarding an ethylene response in a plant or plant part comprising the step of contacting the plant or plant part with an ethylene response retarding amount of benzocyclopropene and/or naphtho[b]cyclopropane.

### TECHNICAL FIELD

A method of retarding an ethylene response in a plant.

### BACKGROUND ART

The following discussion of the background art is intended to facilitate an understanding of the present invention only. The discussion is not an acknowledgement or admission that any of the material referred to is or was part of the common general knowledge as at the priority date of the application.

Amongst different types of foods, fresh horticultural produce is highly perishable and postharvest losses (PHL) are up to 44% (0.57 billion tonnes) globally. Reduction of PHL will not only ensure food and nutritional security to the growing world population but also contribute to, decrease the global warming through reduced use of land, water and other natural resources. The availability of high quality fresh fruits and vegetables at reasonable costs beyond the season could be ensured through the reduction of PHL from the farm gate to the consumers.

Ethylene promotes fruit ripening, senescence and abscission of plant organs and hence plays a key role in causing quantitative and qualitative postharvest losses in fresh horticultural produce. Usage of ethylene antagonists is one of the most effective approaches in retarding fruit ripening, extending postharvest life, maintaining quality and reducing PHL in fresh horticultural produce.

1-Methylcyclopropene (1-MCP) has been used commercially as an ethylene action inhibitor to retard fruit ripening and flower abscission. 1-MCP is also recommended for use in fresh horticultural produce. 1-MCP is a gas at room temperature and is highly unstable and difficult to use. In addition, it is not easily available to growers and is extremely expensive as its treatment is available only as a service not as a chemical.

### SUMMARY OF INVENTION

In accordance with the present invention, there is provided a method for retarding an ethylene response in a plant or plant part comprising the step of contacting the plant or plant part with an ethylene response retarding amount of benzocyclopropene and/or naphtho[b]cyclopropane.

In the context of the present specification, the term plant shall be understood to include whole plants and parts thereof, such as field crops, potted plants, cut flowers and fruits and vegetables.

The present invention can be employed to combat more than one different ethylene response. Ethylene responses may be initiated by either exogenous or endogenous sources of ethylene. Ethylene responses include, for example, the ripening and/or softening of fruits and vegetables, colour loss in vegetables, shattering losses of pods and crop plants, senescence of flowers, abscission of foliage, flowers and fruit, the prolongation of the life of plants such as potted plants, cut flowers and dormant seedlings, the inhibition of growth and the stimulation of growth, adverse effects caused by stress [biotic and abiotic (wounding and mechanical stress, water stress, salinity, flooding/hypoxia, chilling, ozone injury)], degeneration of chlorophyll.

Ethylene responses or ethylene-type responses may also include increasing yields, increasing disease resistance, facilitating interactions with herbicides, increasing resistance to freeze injury, hormone or epinasty effects, hastening ripening and colour promotion in fruit, promotion of sprouting in tubers, abscission of foliage, flowers and fruit, increasing flowering and fruiting, abortion or inhibition of flowering and seed development, increasing yields of plant-based oils and fats, prevention of lodging, stimulation of seed germination and breaking of dormancy, facilitating interactions with other growth regulators, auxin activity, inhibition of terminal growth, control of apical dominance, increase in branching, increase in tillering and changing biochemical compositions of plants.

In accordance with the present invention, there is provided a method for retarding an ethylene response in a plant or plant part comprising the step of contacting the plant or plant part with an ethylene response retarding amount of benzocyclopropene and/or naphtho[b]cyclopropane wherein the ethylene response includes the ripening and/or softening of fruits and vegetables, colour loss in vegetables, reduced browning in minimally produced fruits and vegetables, shattering losses of pods and crop plants, senescence of flowers, abscission of foliage, flowers and fruit, the prolongation of the life of plants such as potted plants, cut flowers and dormant seedlings, the inhibition of growth and the stimulation of growth, adverse effects caused by stress [biotic and abiotic (wounding and mechanical stress, water stress, salinity, flooding/hypoxia, chilling, ozone injury)], degeneration of chlorophyll, increasing disease resistance, facilitating interactions with herbicides, increasing resistance to freeze injury, hormone or epinasty effects, hastening ripening and colour promotion in fruit, promotion of sprouting in tubers, abscission of foliage, flowers and fruit, increasing flowering and fruiting, abortion or inhibition of flowering and seed development, increasing yields of plant-based oils and fats, prevention of lodging, stimulation of seed germination and breaking of dormancy, facilitating interactions with other growth regulators, auxin activity, inhibition of terminal growth, control of apical dominance, increase in branching, increase in tillering and changing biochemical compositions of plants.

The method of the present invention can enable seeds to be stored for longer times without sprouting if treated with benzocyclopropene and/or naphtho[b]cyclopropane prior to storage. Fruit can be maintained on the plant, as can grain and legumes, until harvesting can take place at a later than normal time, either for convenience in harvesting or to extend the growing season.

In accordance with the present invention, there is provided a method of retarding ripening and/or softening of a plant or plant part comprising administering to a plant or plant part a ripening or softening regulating amount of benzocyclopropene and/or naphtho[b]cyclopropane. In one form of the invention, the plant part is a fruit. In a second form of the invention, the plant part is a vegetable.

In accordance with the present invention, there is provided a method for retarding senescence of a plant or plant part comprising the step of contacting the plant or plant part with a senescence retarding amount of benzocyclopropene and/or naphtho[b]cyclopropane.

In accordance with the present invention, there is provided a method for retarding abscission of a plant or plant part comprising the step of contacting the plant or plant part with an abscission retarding amount of benzocyclopropene and/or naphtho[b]cyclopropane.

In accordance with the present invention, there is provided a method for extending the life of a cut plant comprising the step of contacting the plant with a life extending amount of benzocyclopropene and/or naphtho[b]cyclopropane.

The method for extending the life of a cut plant comprising the step of contacting the plant with an effective life extending amount of benzocyclopropene and/or naphtho[b]cyclopropane may include extending the vase life of the cut plant.

In accordance with the present invention, there is provided a method for extending the storage life of fresh horticultural produce comprising the step of contacting the produce with an life extending amount of benzocyclopropene and/or naphtho[b]cyclopropane.

In the context of the present invention, the term ripening shall be understood to encompass ripening of the fruit or vegetable while still on the relevant plant and the ripening after harvest.

In accordance with the present invention, there is provided a method of reducing the rate of germination of a seed comprising applying to a seed or to a plant in which the seed is being formed, a germination reducing amount of benzocyclopropene and/or naphtho[b]cyclopropane.

In accordance with the present invention, there is provided a method of retarding colour loss in vegetables comprising administering to a plant or plant part a regulating amount of benzocyclopropene and/or naphtho[b]cyclopropane.

In accordance with the present invention, there is provided a method of reducing browning in minimally produced fruits and vegetables comprising administering to a plant or plant part a regulating amount of benzocyclopropene and/or naphtho[b]cyclopropane.

In accordance with the present invention, there is provided a method of retarding shattering losses of pods and crop plants of a plant or plant part comprising administering to the plant or plant part a regulating amount of benzocyclopropene and/or naphtho[b]cyclopropane.

In accordance with the present invention, there is provided a method of retarding senescence of flowers comprising administering to a plant or plant part a senescence retarding amount of benzocyclopropene and/or naphtho[b]cyclopropane.

In accordance with the present invention, there is provided a method of retarding abscission of foliage, flowers and fruit of a plant or plant part comprising administering to the plant or plant part a abscission retarding amount of benzocyclopropene and/or naphtho[b]cyclopropane.

In accordance with the present invention, there is provided a method of prolonging of the life of plants such as potted plants, cut flowers and dormant seedlings comprising administering to the plant or plant part a life prolonging amount of benzocyclopropene and/or naphtho[b]cyclopropane.

In accordance with the present invention, there is provided a method of retarding adverse effects caused by stress [biotic and abiotic (wounding and mechanical stress, water stress, salinity, flooding/hypoxia, chilling, ozone injury)], of a plant or plant part comprising administering to the plant or plant part a regulating amount of benzocyclopropene and/or naphtho[b]cyclopropane.

In accordance with the present invention, there is provided a method of increasing disease resistance of a plant or plant part comprising administering to the plant or plant part a disease resistance increasing amount of benzocyclopropene and/or naphtho[b]cyclopropane.

In accordance with the present invention, there is provided a method of increasing resistance to freeze injury, hormone or epinasty effects of a plant or plant part comprising administering to the plant or plant part a regulating amount of benzocyclopropene and/or naphtho[b]cyclopropane.

In accordance with the present invention, there is provided a method of hastening ripening and colour promotion in fruit comprising administering to a plant or plant part a regulating amount of benzocyclopropene and/or naphtho[b]cyclopropane.

In accordance with the present invention, there is provided a method of promotion of sprouting in tubers, comprising administering to a plant or plant part a regulating amount of benzocyclopropene and/or naphtho[b]cyclopropane

In accordance with the present invention, there is provided a method of retarding abscission of foliage, flowers and fruit of a plant or plant part comprising administering to the plant or plant part an abscission retarding amount of benzocyclopropene and/or naphtho[b]cyclopropane.

The step of contacting the plant or plant part with an ethylene response retarding amount of benzocyclopropene and/or naphtho[b]cyclopropane may comprise dipping, spraying, fumigating, irrigating or brushing at least a portion of the plant or plant part with or in a solution or any combination thereof.

Since ethylene acts as a ripening hormone in all known plants, there are no known limits on the plants or plant parts to which this invention can be applied. However, the invention is preferably practiced with agricultural products intended for human consumption and use in which spoilage during storage is a common problem, such as produce and fresh flowers.

Field crops that may be treated by the method of the present invention include cereals including wheat, barley, rice, rye, maize, sorghum and oats, legumes including soybeans, peas, peanuts and beans, oil-producing plants including mustard, canola, sunflower, safflower, castor, flax, sesame, perilla and rape, fibre-producing plants including cotton and hemp, and tobacco.

Fruits that may be treated by the method of the present invention include apple, nectarines, plums, tomatoes, apples, bananas, pears, papaya, mangoes, peaches, apricots, oranges, lemons, limes, grapefruit, tangerines, kiwifruit, pineapple, persimmon, avocados, melons, berries, cherries and other commercial cultivars, hybrids and new developed cultivars.

Vegetables that may be treated by the method of the present invention include leafy green vegetables such as lettuce, spinach and cabbage, roots such as potatoes and carrots, bulbs such as onions and garlic, herbs such as basil, oregano, dill, legumes such as soybean, lima beans and peas and corn, broccoli, cauliflower and asparagus.

Tubers which may be treated by the method of the present invention include potatoes, sweet potatoes, cassava and dahlia.

Ornamental plants which may be treated by the method of the present invention to inhibit senescence and/or to prolong flower life and appearance (e.g., delay yellowing and abscission), include potted ornamentals, and cut flowers. Potted ornamentals and cut flowers which may be treated with the present invention include wax flowers, azalea, hydrangea, hybiscus, snapdragons, poinsettia, cactus, begonias, roses, tulips, daffodils, petunias, carnation, lily, gladiolus, alstroemeria, anemone, columbine, aralia, aster, bougainvillea, camellia, bellflower, cockscomb, falsecypress, chrysanthemum, clematis, cyclamen, freesia, and orchids of the family Orchidaceae and other commercial cultivars, hybrids and new developed cultivars.

Plants which may be treated by the method of the present invention include, apples, pears, mangos, cherries, pecans, grapes, olives, coffee, snapbeans, oranges, lemons, limes, grapefruit, tangerines and other commercial cultivars, hybrids and new developed cultivars, and weeping fig, as well as dormant seedlings such as various fruit trees including apple, ornamental plants, shrubbery, and tree seedlings.

In addition, shrubbery which may be treated according to the present invention to inhibit abscission of foliage include privet, photinea, holly, ferns, aglaonema, cotoneaster, barberry, waxmyrtle, abelia, acacia and bromeliades of the family Bromeliaceae, and other commercial cultivars, hybrids and new developed cultivars.

Fibre and oil seed crops which may be treated by the method of the present invention to inhibit abscission include cotton balls and seed shattering from pods in rapeseed, mustard and canola crops.

Plant-based oils and fats include vegetable oils such as palm, coconut, canola, soy bean, sunflower, peanut, hazelnut, rapeseed, cottonseed, olive, corn, grapeseed, linseed, sassflower and sesame.

The benzocyclopropene and/or naphtho[b]cyclopropane may be applied to the plant or plant part at any time, depending on the nature of the ethylene response. When used to control senescence or abscission, the benzocyclopropene and/or naphtho[b]cyclopropane are preferably applied five weeks prior to harvest. However, earlier and later applications can still be effective. It will be appreciated that the timing of application will be influenced by the species of plant and the nature of the ethylene reponse. Preferably, the benzocyclopropene and/or naphtho[b]cyclopropane are applied two to three days before ethylene peaking.

It will be appreciated that the benzocyclopropene and/or naphtho[b]cyclopropane may be applied to the plant or plant part more than once.

The benzocyclopropene and/or naphtho[b]cyclopropane may be applied to the plant or plant part prior to harvest, post-harvest or both.

A preferred method of application is by fumigation in a closed environment wherein a delivery substrate is contacted with a solution of benzocyclopropene and/or naphtho[b]cyclopropane and placed in a closed environment with the plant or plant part. The solution of benzocyclopropene and/or naphtho[b]cyclopropane preferably comprises a water/alcohol mixture. Evaporation of the solvent facilitates vaporisation of the benzocyclopropene and/or naphtho[b]cyclopropane and subsequent distribution throughout the closed environment. Evaporation and vaporisation may be facilitated by a fan or the like.

The delivery substrate may be plastic, paper or fabric from natural or synthetic fibres. Preferably, the delivery substrate is a filter paper.

The delivery substrate may further comprise a release agent to facilitate the delivery of the benzocyclopropene and/or naphtho[b]cyclopropane such as hydroxypropylmethylcellulose or polyvinylpyrrolidone.

The amount of the active ingredient in the form of benzocyclopropene and/or naphtho[b]cyclopropane required to inhibit the ethylene response will vary depending on numerous factors including the type of active ingredient, the type of ethylene response and the genotype and amount of plant material as well as the method of application.

For a given species and ethylene response to be retarded, the required dosage and treatment regime can be readily determined by carrying out appropriate experiments as described herein.

Preferably, the step of administering the benzocyclopropene and/or naphtho[b]cyclopropane to a surface of the plant or plant part comprises applying the composition at a rate of from 0.01 to 100 ppm active ingredient based on the weight of the plant or plant part to which the composition is applied.

For fumigation purposes, a solution concentration would range from 0.01 nLL⁻¹ to 1000 μLL⁻¹ (v/v). More preferably, 1 nLL⁻¹ to 1000 nLL⁻¹. More preferably, 10 nLL⁻¹ to 100 nLL⁻¹. More preferably, 50 nLL⁻¹ to 100 nLL⁻¹.

Solutions for direct application such as spraying, dipping, waxing and irrigation, use as foliar sprays or irrigation may be prepared at concentrations up to 5000 mgL⁻¹. Preferably, solution concentrations range from 0.1 mgL⁻¹ to 1000 mgL⁻¹. More preferably, 1 mgL⁻¹ to 100 mgL⁻¹. More preferably, 10 mgL⁻¹ to 50 mgL⁻¹.

Preferably, solutions of benzocyclopropene and/or naphtho[b]cyclopropane are sprayed until run off.

Regardless of the method of applications, concentrates of benzocyclopropene and/or naphtho[b]cyclopropane may be prepared for later dilution and application.

The benzocyclopropene and/or naphtho[b]cyclopropane may be applied in solid forms, for example, for dusting.

Dusts may be prepared with known excipients as described herein.

The method of the present invention may also include the application of benzocyclopropene and/or naphtho[b]cyclopropane in packaging material or in the space in packaged produce. For example, packaging film such as polyvinyldene chloride or low density polyethylene may have benzocyclopropene and/or naphtho[b]cyclopropane impregnated into the film. Alternatively, the air space inside the packaging may comprise benzocyclopropene and/or naphtho[b]cyclopropane.

In accordance with the present invention, there is provided a composition for retarding an ethylene response in a plant or plant part comprising an ethylene response retarding amount of benzocyclopropene and/or naphtho[b]cyclopropane.

The composition may be provided in the form of wettable powders, dusting powders, solutions, emulsifiable concentrates, emulsions, suspension concentrates, and aerosols.

Where the composition is a solution, the composition preferably comprises a substantially aqueous solution or organic solvent or a combination of both.

In one form of the invention, the organic solvent is an alcohol. Preferably, the alcohol is ethanol.

The composition may comprise one or more agronomically acceptable adjuvants such as carriers, extenders, binders, lubricants, surfactants, dispersants, wetting agents, spreading agents, dispersing agents, stickers, adhesives, defoamers, thickeners and emulsifying agents.

A carrier may be provided in the form of an inorganic or an organic material. The carrier may be a liquid such as an organic solvent such as hydrocarbons and alcohols. Preferred examples include cyclodextrin and polyoxyethylenes such as Tween20. Alternatively, the carrier may be in solid form such as talc or other inorganic, substantially inert materials, such as clays or zeolites.

Surface active agents such as emulsifiers, dispersing agents, wetting agents may be ionic or non-ionic. Wetting agents may include various alkyl aryl sulfate salts, alkyl aryl sulfonate salts, polyalkyl alcohols, polyacrylic acids; salts of ligninsulphonic acids; condensates of ethylene oxide with fatty alcohols, fatty acids or fatty amines; fatty acids; fatty amines.

Where the composition is provided as a wettable powder, the composition preferably comprises about 0.1-99% ^{w}/_{w} of benzocyclopropene and/or naphtho[b]cyclopropane

In one form of the invention, the composition comprises about 0.1-99% ^{w}/_{w} of benzocyclopropene and/or naphtho[b]cyclopropane In an alternate form of the invention, the composition comprises about 40-70% ^{w}/_{w} of benzocyclopropene and/or naphtho[b]cyclopropane In an alternate form of the invention, the composition comprises about 50-60% ^{w}/_{w} of benzocyclopropene and/or naphtho[b]cyclopropane In an alternate form of the invention, the composition comprises about 1-90% ^{w}/_{w} of benzocyclopropene and/or naphtho[b]cyclopropane In an alternate form of the invention, the composition comprises about 1-50% ^{w}/_{w} of benzocyclopropene and/or naphtho[b]cyclopropane In an alternate form of the invention, the composition comprises about 1-20% ^{w}/_{w} of benzocyclopropene and/or naphtho[b]cyclopropane In an alternate form of the invention, the composition comprises about 1-10% ^{w}/_{w} of benzocyclopropene and/or naphtho[b]cyclopropane In an alternate form of the invention, the composition comprises about 1-5% ^{w}/_{w} of benzocyclopropene and/or naphtho[b]cyclopropane In an alternate form of the invention, the composition comprises about 5% ^{w}/_{w} of benzocyclopropene and/or naphtho[b]cyclopropane

The composition may further comprise about 0-5% ^{w}/_{w} of a wetting agent.

The composition may further comprise about 0-10% ^{w}/_{w} of a dispersing agent.

The composition may further comprise about 0-10% ^{w}/_{w} of one or more stabilizers and/or other adjuvants, such as penetrating agents, adhesives, anticaking agents, dyestuffs, and the like.

Particular materials which may find use include sodium isopropylnaphthalenesulfonate, sodium naphthalenesulfonate, sodium dodecylbenzenesulfonate, oxyethyleneated alyklphenol (where the alkyl group is from about 8 to 18 carbon atoms and the number of oxyethylene groups will vary from about 1 to 50, usually from about 5 to 20), silica, kaolin, Benomyl wettable powder, and the like.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is the chemical structures of benzocyclopropene and naphtho[b]cyclopropane;

FIG. 2 depicts the concentration of ethylene and CO₂ on the day of climacteric peak;

FIG. 3 depicts the concentration of ethylene (A) and CO₂ (B) on the day of climacteric peak in ‘Fortune’ plum fruit treated with different concentration of naphtho[b]cyclopropane (NC); and

FIG. 4 depicts the percent of flower/bud abscission after two days of treatment with antagonist.

### DESCRIPTION OF EMBODIMENTS

Throughout this specification, unless the context requires otherwise, the word “comprise” or variations such as “comprises” or “comprising”, will be understood to imply the inclusion of a stated integer or group of integers but not the exclusion of any other integer or group of integers.

Benzocyclopropene was prepared as shown in Scheme 1.

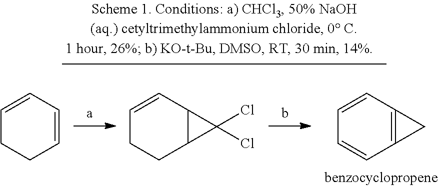 [SMILES: C1=CCCC=C1 ; ClC1(Cl)C2C=CCCC21 ; c1ccc2c(c1)C2]

A solution of 1,3-cyclohexadiene (80.5 g, 0.5 mol), cetyltrimethylammonium bromide (2.00 g) in 50% aqueous sodium hydroxide (200 g) was cooled (0-25° C.) and stirred under nitrogen. Ethanol (5 mL) and chloroform (80 mL) were added successively in one portion. The solution was stirred for 1 hour at 0° C. and allowed to warm to room temperature for a further 1 hour. Water was added to the reaction mixture and extracted. The organic phase was washed with water (2×100 mL), dried (CaCl₂) and concentrated under reduced pressure to provide an oil. The oil was purified by flash chromatography to afford the 7,7-dichlorobicyclo[4.1.0]-hept-2-ene as a colourless oil (42 g, 26%). The NMR spectroscopic data was identical to those reported.

Potassium t-butoxide was added in portions to a solution of 7,7-dichlorobicyclo[4.1.0]-hept-2-ene (1.00 g, 6.1 mmol) in anhydrous DMSO (30 mL) under nitrogen. The dark brown mixture was stirred for 30 minutes. A vacuum was applied to the reaction mixture and the volatiles collected in an −86° C. trap. The distillate was diluted in petroleum spirits, and washed with brine (4×60 mL) and water (2×30 mL), dried (Na₂SO₄) and concentrated under reduced pressure at 0° C. to afford benzocyclopropene as an oil (80 mg, 14%). ¹H NMR (CDCl₃) δ 3.17 (2H, s), 7.21 (s, 4H). The NMR spectroscopic data was identical to those reported.

Naphtho[b]cyclopropene was prepared as shown in Scheme 2.¹ ¹W. E. Billups and C. Y. Chow J. Am. Chem. Soc. 1973, 95, 4099.

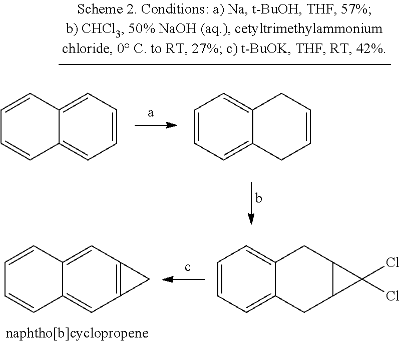 [SMILES: c1ccc2ccccc2c1 ; C1=CCc2ccccc2C1 ; c1ccc2cc3c(cc2c1)C3 ; ClC1(Cl)C2Cc3ccccc3CC21]

Small pieces of sodium metal (15 g) were added to solution of naphthalene (30 g) in anhydrous THF (100 mL). The solution turned to a deep green colour during this time. A solution of t-butanol (24 mL) and THF (24 mL) in water was added dropwise over 20 minutes. The resulting solution was stirred for a further 3 hours. The excess sodium metal was removed by filtration and the filtrate washed with water (2×50 mL), dried and concentrated under reduced pressure to give pure 1,4-dihydronaphthalene as a colourless solid (17.5 g, 57%). The NMR spectroscopic data was identical to those reported.

A solution of 1,4-dihydronaphthalene (17.0 g, 0.131 mol), cetyltrimethylammonium bromide (0.567 g, 1.6 mmol) in 50% aqueous sodium hydroxide (50 g) was cooled (0-25° C.) and stirred under nitrogen. Ethanol (1.6 mL) followed by chloroform (23 mL) was added. The solution was stirred for 1 hour at 0° C. and allowed to warm to room temperature for a further 1 hour. Water (100 mL) was added to the reaction mixture and extracted. The organic phase was washed with water (2×50 mL), dried (CaCl₂) and concentrated under reduced pressure to provide an oil. The oil was purified by flash chromatography to afford the adduct (7.56 g, 27%). The NMR spectroscopic data was identical to those reported.

Potassium t-butoxide (11.0 g, 98.2 mmol) was added in portions to a solution of 1,1-dichloro-1a,2,7,7a-tetrahydro1H-cyclopropa[b]naphthalene (4.78 g, 29.3 mmol) in anhydrous THF (60 mL) under nitrogen at room temperature and stirred for a further 18 hour. The resulting mixture was diluted in petroleum spirits (20 mL), washed with brine (4×20 mL) and water (2×10 mL), dried (Na₂SO₄) and concentrated under reduced pressure. The residue was purified by flash chromatography to afford naphtho[b]cyclopropene as a colourless solid (1.11 g, 42%). The NMR spectroscopic data was identical to those reported.

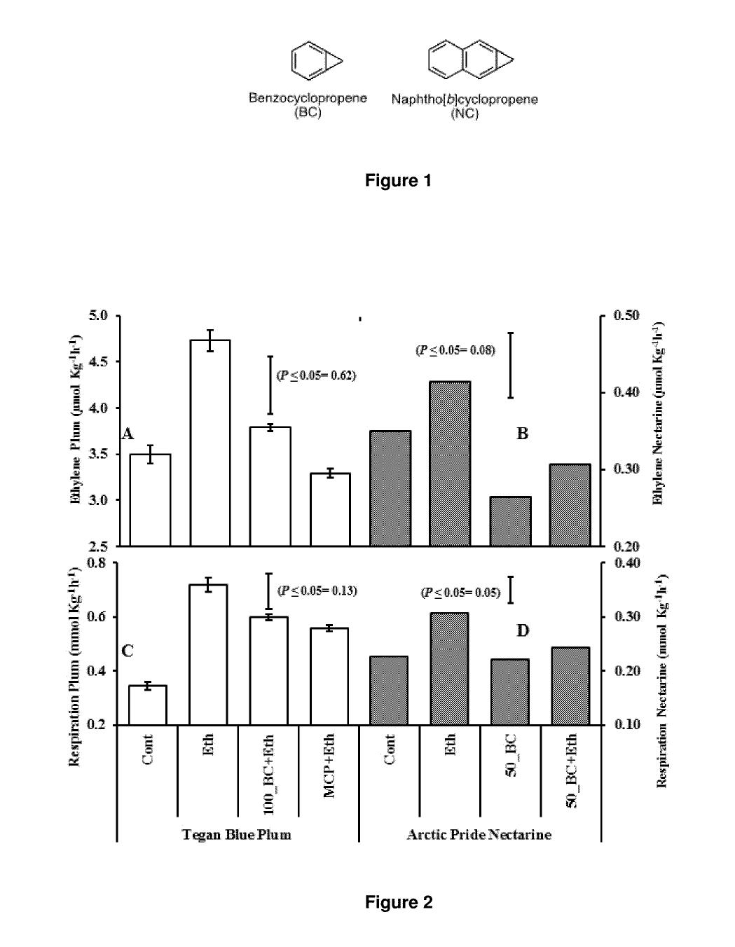

The chemical structures of benzocyclopropene (BC) and naphtho[b]cyclopropene (NC) are provided at FIG. 1. Advantageously, benzocyclopropene is a liquid at room temperature, making it easier to handle than 1-methylcyclopropene. Advantageously, naphthocyclopropene is a solid at room temperature, making it easier to handle than 1-methylcyclopropene.

Advantageously, benzocyclopropene and naphtho[b]cyclopropane are stable at room temperature for several months.

Benzocyclopropene and naphtho[b]cyclopropene are only partially soluble in water. To prepare substantially aqueous solutions of these compounds, the lead compounds were dissolved in ethanol and then diluted with water.

Various experiments were conducted using ‘Tegan Blue’ and ‘Fortune’ Japanese plums, ‘Arctic Pride’ nectarine, Fuji and Pink Lady™ apples, WX7, WX17, WX39, WX 56, WX 58, WX73 and WX107, WXFU, hybrid, Revelation, Purple Pride and Jenny wax flowers (Chamelaucium Desf.).

Mature ‘Tegan Blue’ and ‘Fortune’ Japanese plum and ‘Arctic Pride’ nectarine fruits of uniform size and maturity, free from visual blemishes and diseases were harvested in early morning from a commercial orchard in Western Australia. Following the harvest, the fruit were brought to the Horticulture Research Laboratory, Curtin University, using a temperature controlled vehicle at 20-25° C. The fruit were treated by fumigation with 0 to 100 nLL⁻¹ of BC and 1000 nLL⁻¹ of 1-MCP (Tegan Blue plum) for 18 hr at ambient conditions (20±1° C. and 65±5% RH) by using hermetically sealed plastic containers of 60 L volume. Whatman filter paper (number 2) soaked with specific concentrations of BC and 1-MCP were kept along with the fruit and 30 g of soda lime inside each container. A small battery operated fan was used to ensure equal distribution of the vapours from the chemicals. Half of the treated fruit was exposed to ethylene (10 μL L⁻¹) for 24 hr following BC and 1-MCP treatment.

In a second set of experiments, ‘Fortune’ plum fruit were treated with 0 to 1000 nIL⁻¹ of NC as described above. The treated fruit were kept in the ambient conditions for ripening and endogenous level of ethylene and CO₂ was determined. The experiments were laid out by following completely randomized design (CRD) with four replications for each treatment and 10 fruit in each replication.

The endogenous level of ethylene was determined by using the Sensor Sense (Sensor sense B. V, Nijmegen, The Netherlands). The Sensor Sense includes an ETD 300 ethylene detector, a set of valve controllers with an option of six valves connected to six separate cuvettes [1.0 L air-tight jar, fitted with a rubber septum (SubaSeal®, Sigma-Aldrich Co., St. Louis, USA)]. The continuous flow method was used with coarse mode (conversion factor 99818, capacity to measure ethylene concentration at 0-500 ppm, sensitivity at <1%) of analysis. Each sample was run for 20 minutes with a flow rate of 4.0 L hour⁻¹ and the average reading of last 15 minutes was considered to calculate the concentration of ethylene and expressed as μmol kg⁻¹ kg⁻¹ h⁻¹.

Respiration rate was determined as carbon dioxide (CO₂) production from the fruit during ripening period a using CO₂ analyser. The headspace gas sample (2.0 mL) was taken through rubber septum (SubaSeal®, Sigma-Aldrich Co., St. Louis, USA) using a syringe from the air tight jar with sample fruit and injected into an infrared gas analyser [Servomex Gas Analyzer, Analyzer series 1450 Food Package Analyzer, Servomex (UK) Ltd., East Sussex, UK]. The respiration rate was calculated on the basis of the peak areas of 2.0 mL gas sample and CO₂ standard (8.52±0.17%) and expressed as mmoL CO₂ kg⁻¹ kg⁻¹ h⁻¹.

To evaluate the effects of BC and NC on flower abscission, flowering stems of Wax flower (Chamelaucium Desf.) (WX17, WX73 and WX107) were collected from mature bushes grown at Department of Agriculture and Food Western Australia (DAFWA), Perth (31° 58′ 55″ S/115° 51′ 47″ E). Collected stems were immediately placed upright in buckets with water and recut at 20-25 cm in length (from the cut end to the most extreme opened-flowers). The flower stalks were treated similarly as the fruits with BC (0-100 nLL⁻¹) or NC (0-100 nLL⁻¹) and ethylene. The experiments were laid out by following CRD design, having five replications for each treatment and five stalks in each replication. During the treatment period, the flower stalks were placed in small plastic bottles with distilled water. A cone made of nylon mesh was placed at the base of the stalks to check the number of abscised flowers.

Assessment of floral organs abscission (%): Following ethylene treatment (2-4 days), the flower stalks were taken out from the treatment container and gently beaten against a collection tray to calculate the percentage of abscised flowers and buds.

The experimental data were analysed following one-way analysis of variance (ANOVA) by using Genstat 13 (release 13.1; Lawes Agricultural Trust, Rothamsted Experimental Station, Harpenden, UK). The effects of various treatments and their interactions were assessed and least significant differences (Fisher's LSD) were calculated by F test at 5% level of significance.

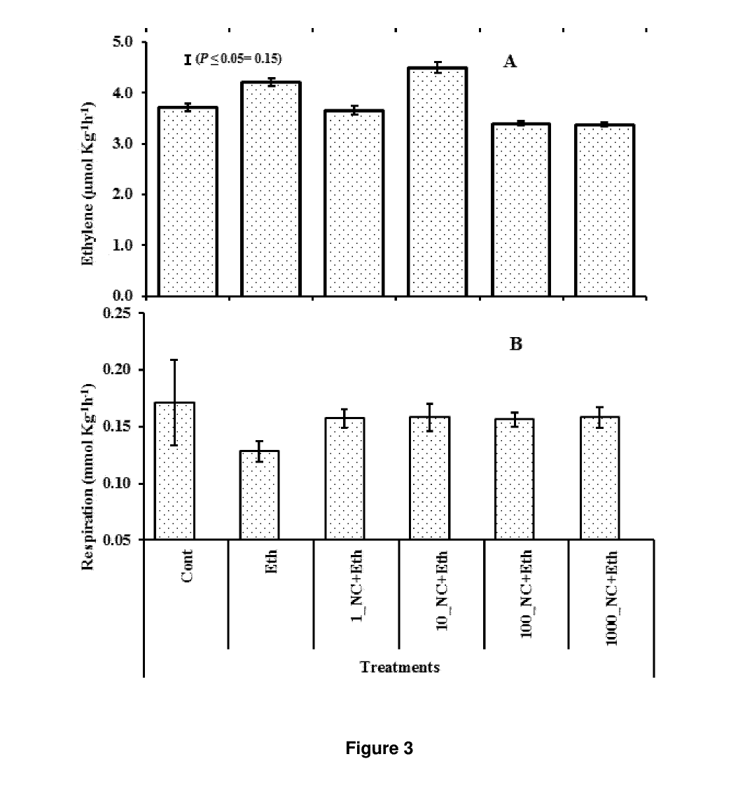

The level of climacteric ethylene in ‘Tegan Blue’ plum fruit was significantly (P≤0.05) suppressed by 100 nLL⁻¹ BC+ethylene and 1000 nLL⁻¹ 1-MCP+ethylene (0.80- and 0.70-fold respectively) in comparison to the solely ethylene treated fruit where the ethylene concentration was 4.73 μmol kg⁻¹ kg⁻¹ h⁻¹ (FIG. 2A).

Similarly, BC (50 nLL⁻¹)+ethylene treated ‘Arctic Pride’ nectarine fruit exhibited significantly suppressed (0.63-fold) levels of ethylene than the solely ethylene treated fruit (0.414 μmol kg⁻¹ kg⁻¹ h⁻¹) (FIG. 2 B).

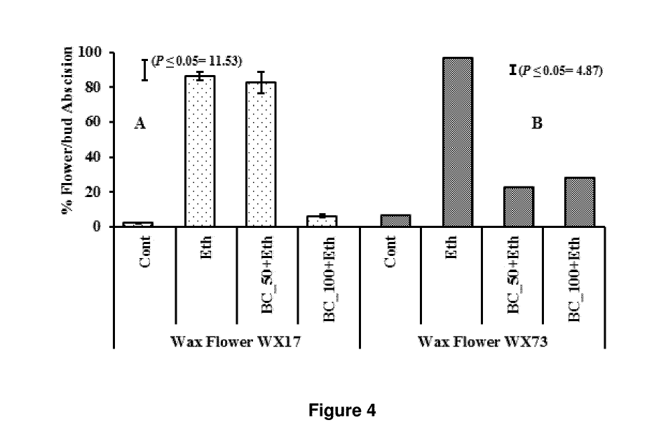

The NC (100-1000 nLL-1) also showed antagonistic effect by significantly suppressing the level of climacteric ethylene (0.81-fold) than the solely ethylene treated fruit in ‘Fortune’ plum fruit (FIG. 3).

The climacteric respiration was also suppressed in BC (100 nLL⁻¹)+ethylene and 1-MCP (1000 μLL⁻¹)+ethylene treated ‘Tegan Blue’ plum fruit (0.83- and 0.77-fold respectively) than the solely ethylene treated fruit (0.72 mmol CO₂ kg⁻¹ kg⁻¹ h⁻¹) (FIG. 2C). On the other hand, significant suppression of respiration climacteric was observed in both BC (50 nLL⁻¹) and BC (50 nLL⁻¹)+ethylene treated ‘Arctic Pride’ nectarine fruit (0.71- and 0.77-fold respectively) in comparison to the solely ethylene treated fruit (0.31 mmol CO₂ kg⁻¹ kg⁻¹ h⁻¹) (FIG. 2D).

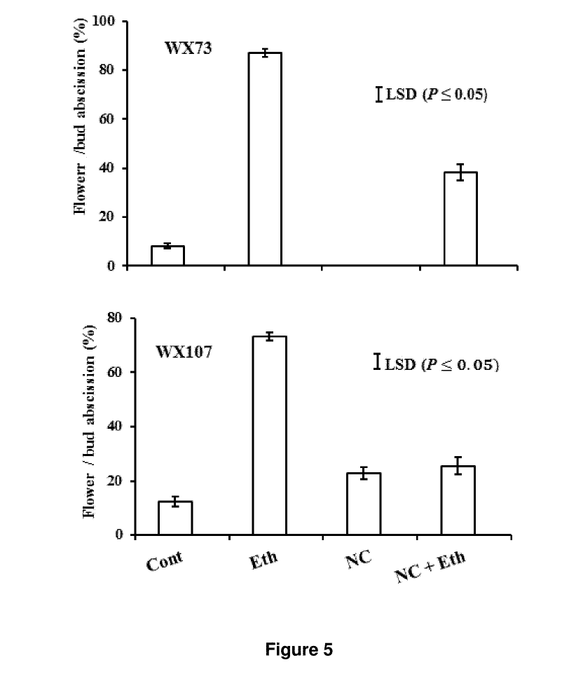

The fumigation of BC (100 nLL⁻¹) followed by ethylene exposure (10 μLL⁻¹) significantly reduced the rate of flower/bud abscission in WX17 (6.05%). Whilst 50 and 100 nL L⁻¹ BC followed by ethylene treatment significantly lowered the rate of abscission at 22.43% and 28.40% respectively in WX73 wax flower as compared to ethylene treatment alone (FIGS. 4 A and B).

The treatment of NC (100 nL L⁻¹) also significantly (P 0.05) suppressed the rate of flower/bud abscission in WX73 (0%) and WX107 (22.82%) wax flowers in comparison to the ethylene treated flowers. Suppressed flower/bud abscission was also observed in WX73 and WX107 wax flowers (38.11% and 25.51% respectively), even when the NC treatment was followed by ethylene treatment (10 μL L⁻¹) (FIG. 5). The highest level of flower/bud abscission in all genotypes was noted from the ethylene treated flowers.

Fruit and flower stalks treated with BC or NC (50-100 nL L⁻¹) followed by ethylene treatment (10 μL L⁻¹) significantly (P≤0.05) reduced the rate of flower abscission and concentration of climacteric ethylene and CO₂ production than the solely ethylene exposed flowers and fruits which suggests that the inhibition of ethylene action by the BC and NC was not only exogenous but also at endogenous. This is the first disclosure on the effects of BC and NC on antagonising ethylene action during fruit ripening and floral organs abscission processes.

Similar effects to 1-MCP have been observed for BC and NC which has been reflected through the non-significant differences among the effects of BC (100 nL L⁻¹) and 1-MCP (1000 nL L⁻¹) on climacteric ethylene in ‘Tegan Blue’ and ‘Fortune’ Japanese plum fruits (FIGS. 2 A and 3 A) ‘Arctic Pride’ nectarine (FIG. 2 B) and respiration in ‘Tegan Blue’ plum (FIG. 2C) and ‘Arctic Pride’ nectarine (FIG. 2 D).

BC and NC fumigation exhibited ethylene antagonistic effects on ripening of climacteric fruits such as plums, nectarines and abscission of floral organs in wax flowers.

In the tested chemicals (BC and NC), the cyclopropene portion of the molecule is thought to make a potential bond at or near the ethylene binding site of the receptor. As the flowers/fruit were exposed to the BC and NC treatment shortly after collection and completely blocked the ethylene receptor to prohibit ethylene activity for a period of time, so it worked at a non-competitive basis.

1. A method for retarding an ethylene response in a plant or plant part comprising the step of contacting the plant or plant part with an ethylene response retarding amount of benzocyclopropene and/or naphtho[b]cyclopropane.

2. The method for retarding an ethylene response in a plant or plant part in accordance with claim 1, wherein the ethylene response is selected from the ripening and/or softening of fruits and vegetables, colour loss in vegetables, reduced browning in minimally produced fruits and vegetables, shattering losses of pods and crop plants, senescence of flowers, abscission of foliage, flowers and fruit, the prolongation of the life of plants such as potted plants, cut flowers and dormant seedlings, the inhibition of growth and the stimulation of growth, adverse effects caused by stress [biotic and abiotic (wounding and mechanical stress, water stress, salinity, flooding/hypoxia, chilling, ozone injury)], degeneration of chlorophyll, increasing yields, increasing disease resistance, facilitating interactions with herbicides, increasing resistance to freeze injury, hormone or epinasty effects, hastening ripening and colour promotion in fruit, abscission of foliage, flowers and fruit, increasing flowering and fruiting, abortion or inhibition of flowering and seed development, prevention of lodging, stimulation of seed germination and breaking of dormancy, facilitating interactions with other growth regulators, auxin activity, inhibition of terminal growth, control of apical dominance, increase in branching, increase in tillering and changing biochemical compositions of plants.

3. The method for retarding an ethylene response in a plant or plant part in accordance with claim 1, wherein the step of contacting the plant or plant part with an ethylene response retarding amount of benzocyclopropene and/or naphtho[b]cyclopropane comprises dipping, spraying, irrigating or brushing at least a portion of the plant or plant part with or in a solution.

4. The method for retarding an ethylene response in a plant or plant part in accordance with claim 1, wherein the plant or plant part is an agricultural product intended for human consumption and use in which spoilage during storage is a common problem, such as produce and fresh flowers.

5. The method for retarding an ethylene response in a plant or plant part in accordance with claim 1, wherein the benzocyclopropene and/or naphtho[b]cyclopropane is applied two to three days before ethylene peaking.

6.-13. (canceled)

14. The method for retarding an ethylene response in a plant or plant part in accordance with claim 1, wherein the benzocyclopropene and/or naptho[b]cyclopropane is applied prior to harvest and/or post-harvest.

15. The method for retarding an ethylene response in a plant or plant part in accordance with claim 1, wherein the benzocyclopropene and/or naptho[b]cyclopropane is applied more than once.

16. The method for retarding an ethylene response in a plant or plant part in accordance with claim 1, wherein the benzocyclopropene and/or naphtho[b]cyclopropane is applied by fumigation in a closed environment.

17. The method for retarding an ethylene response in a plant or plant part in accordance with claim 16, wherein a delivery substrate is contacted with a solution of benzocyclopropene and/or naphtho[b]cyclopropane and placed in the closed environment with the plant or plant part.

18. The method for retarding an ethylene response in a plant or plant part in accordance with claim 17, wherein the delivery substrate is plastic, paper or fabric from natural or synthetic fibres.

19. The method for retarding an ethylene response in a plant or plant part in accordance with claim 17, wherein the delivery substrate is a filter paper.

20. The method for retarding an ethylene response in a plant or plant part in accordance with claim 17, wherein the delivery substrate comprises a release agent to facilitate the delivery of the benzocyclopropene and/or naptho[b]cyclopropane.

21. The method for retarding an ethylene response in a plant or plant part in accordance with claim 1, wherein the plant or plant part is contacted with benzocyclopropene and/or naphtho[b]cyclopropane at a rate of from 0.01 to 100 ppm benzocyclopropene and/or naphtho[b]cyclopropane based on the weight of the plant or plant part.
